(19) United States
(12) Patent Application Publication
(10) Pub. No.: US 2022/0409827 A1
(43) Pub. Date: Dec. 29, 2022

(54) MANAGED DISPENSING OF AEROSOL

(71) Applicant: BOA Nutrition, Inc., Raleigh, NC (US)

(72) Inventors: Hank Durschlag, Raleigh, NC (US); Jon Pritchett, Raleigh, NC (US)

(21) Appl. No.: 17/839,123

(22) Filed: Jun. 13, 2022

Related U.S. Application Data
(60) Provisional application No. 63/209,758, filed on Jun. 11, 2021.

(51) Int. Cl. A61M 11/00

(52) U.S. Cl. CPC A61M 11/001; A61M 11/007

(57) ABSTRACT

Disclosed is an oral mucosa delivery system that allows a composition housed therein to be dispensed in different amount and/or for different duration. The delivery system may comprise a spray cap or an aerosol cap capable of controlling the delivery duration and/or amount by a mechanical or electronic means.

### CROSS REFERENCE TO RELATED APPLICATIONS

The present application claims the priority benefit of U.S. provisional patent application No. 63/209,758 filed Jun. 11, 2021, the disclosure of which is incorporated by reference herein.

### BACKGROUND OF THE INVENTION

### 1. Field of the Disclosure

The present disclosure is generally related to oral mucosa delivery of a composition, specifically the delivery system, kit and use thereof.

### 2. Description of the Related Art

Athletes may experience excessive fluid loss during intense exercise, which leave them with acute electrolyte deficiency in addition to dehydration. Low levels of electrolytes in the system can lead to cramping. To overcome the above issues, athletes may be given oral salt pills to compensate the acute electrolyte and salt loss. This approach may result in the over-supply of sodium. It also has the disadvantage of slow absorption through the GI tract. A mechanism for delivering a smaller dose of electrolytes to the buccal mucosa may provide the necessary electrolytes with more rapid bioavailability.

During exercise, the body requires more oxygen than when the body is at rest. When oxygen cannot be delivered to the muscles faster to replace the used oxygen, the muscles will begin to convert the available glucose to lactic acid. This will build up in the muscle, causing fatigue. Rapid delivery of oxygen to the muscles is thus desired to reduce fatigue.

It has been demonstrated that 100% oxygen administration increased mental and physical performance, such as decreasing reaction times and improving fatigue recovery. The delivery of 100% oxygen is difficult without sealed masks, making it impractical during athletic events. Recent studies have shown that a relatively low concentration of 30-40% hyperoxic air may deliver a similar benefit as 100% oxygen. Therefore, it is desirable to have a system for delivering hyperoxic air to athletes in a convenient fashion.

An athlete's needs for hydration and oxygen may vary greatly depending on the athlete's age, weight, fitness level, type of athletic activity. For example, a person may have a greater need for oxygen when at a high altitude. Therefore, it is desirable to have a delivery system capable of fine tuning the dose and/or dosing frequency according to the athlete's needs.

### SUMMARY OF THE CLAIMED INVENTION

The present invention provides an oral mucosa delivery system capable of delivery a composition in different doses and/or with different dosing frequencies. The present invention also presents the kits and methods of use thereof. In one embodiment, the delivery system may comprise a canister for housing the composition and a cap for dispensing the composition. In one embodiment, the cap is a pump spray cap. In another embodiment, the cap is an adjustable dose pump spray cap. In yet another embodiment, the cap allows to be pressed to different depths, effectuated by a mechanical means attached to the cap. In yet another embodiment, when the cap is pressed to different depths, different dose amount of the composition is drawn to the cap and sprayed out of the cap through a nozzle. In one embodiment, the mechanical means attached to the cap comprises an extension, a collar, and/or at least one receiver.

In one embodiment, the delivery system may comprise a canister for housing the composition and a cap for dispensing the composition. In one embodiment, the cap is a powered aerosol spray cap. The aerosol spray cap may be in any form, shape or configuration capable of dispensing a composition. The composition may be stored in a canister under pressure. The cap may comprise a valve and an outlet. When valve is pressed, the composition may be dispensed through the outlet. In one embodiment, the system may include an actuator that are configured to press the valve when a prompt is received. Such an actuator may comprise an actuator control unit, which may comprise a power source for the actuator, a timer, and an interface that may adjust the time and/or frequency that the valve is pressed. In one embodiment, the actuator control unit may be connected to a smart device to receive recommended dose and dosing information from a hydration app.

In some embodiments, kits and methods of using the above systems are provided. In one embodiment, the method comprises obtaining a canister for housing a composition, connecting the canister to an adjustable spray cap, comprising an extension, a rotatable collar hosting at least one receiver; rotating the collar so that the extension is aligned with the desired receiver; pressing the cap so as to dispense a dose of the composition to the oral mucosa.

### BRIEF DESCRIPTIONS OF THE DRAWINGS

FIG. 1 illustrates an adjustable dose pump spray cap, according to an embodiment of the present invention.

FIG. 2 illustrates a powered aerosol spray cap, according to an embodiment of the present invention.

### DETAILED DESCRIPTION

The present invention provides an oral mucosa delivery system capable of delivery a composition in different doses and/or with different dosing frequencies. The present invention also presents the kits and methods of use thereof. The delivery system may be suitable for delivering athletic supplements in a predetermined dose and/or dosing frequency. The disclosed system is capable of the quick mucosa delivery of nutritions, and may reduce first-pass effects. The instant system may provide a longer duration of nutrient release than traditional oral delivery, such as sports drinks or other orally ingested nutritional supplements.

The delivery system may comprise a canister for housing the composition and a cap for dispensing the composition. In one embodiment, the cap is a pump spray cap. In another embodiment, the cap is an adjustable dose pump spray cap. In yet another embodiment, the cap allows to be pressed to different depths, effectuated by a mechanical means attached to the cap. In yet another embodiment, when the cap is pressed to different depths, different dose amount of the composition is drawn to the cap and sprayed out of the cap through a nozzle. In one embodiment, the mechanical means attached to the cap comprises an extension, a collar, and/or at least one receiver. The cap may have an extension, and the extension may be a ridge that is a part of the adjustable dose pump spray cap. In another embodiment, the system may comprise a collar situated between the adjustable dose pump spray cap and the canister. In another embodiment, the collar and the cap may rotate independently with one another. In yet another embodiment, the rotation may allow the extension to align with the at least one receiver. In one embodiment, the alignment may restrict the depth of the cap can be pressed, and thus limit the dose of the composition dispensed from the cap. In one embodiment, such restriction is enabled through a physical setup inside the at least one receiver. In yet another embodiment, three receivers are present, so at least three different dose amounts can be dispensed by pressing the cap. The system may further comprise sensors, smart phones or smart devices, wherein environmental or user's physiological characteristics are collected, analyzed and/or communicated to the user in real time. Such characteristics can be used to generate recommended dose and dosing frequency information. Such information may guide the user to select the receiver, and thus dispense the recommended dose and/or with the recommended dosing frequency.

The delivery system may comprise a canister for housing the composition and a cap for dispensing the composition. In one embodiment, the cap is a powered aerosol spray cap. The aerosol spray cap may be in any form, shape or configuration capable of dispensing a composition. The composition may be stored in a canister under pressure. The cap may comprise a valve and an outlet. When valve is pressed, the composition may be dispensed through the outlet. In one embodiment, the composition may be dispensed at a constant and predictable rate when the valve is pressed. In one embodiment, the outlet may be shaped to fit in an user's mouth. In one embodiment, the outlet may be designed to dispense the composition towards an user's buccal mucosa. The powered aerosol cap may be affixed to any aerosol valves. Embodiments may include an actuator that are configured to press the valve when a prompt is received. Such an actuator may comprise an actuator control unit. Such actuator control unit may have a power source for the actuator, a timer, and an interface that may adjust the time and/or frequency the valve is pressed. In one embodiment, the actuator control unit may be connected to a computing device, such as a smart phone or a smart device, to allow the user to receive recommended dose and dosing information from a hydration app. In one embodiment, an interface may be built into the cap that allow the user to select the desired dose amount of the composition.

The composition delivered by the instant system may comprise a nutraceutical, a pharmaceutical, a food composition, a food supplement or a nutritional supplement. In one embodiment, the composition may include any one or any combinations of comprise any one or more of (i) a nutritional supplement matrix fraction, (ii) a gas fraction, (iii) an enhancer fraction, (iv) a liquid fraction, or (v) a preservative fraction. The nutritional supplement matrix fraction, the enhancer fraction, the liquid fraction, and the preservation fraction may be all mixed or combined and stored separately from the gas fraction in a canister before being dispensed. Such composition may allow oxygen suspended therein for a specific duration of time before being dispensed from a canister. Furthermore, the composition may be in a compressed state in a canister before being dispensed. The composition may be atomized before or after being dispensed, in order to maximize mucosal absorption.

Furthermore, the composition may be in a compressed state in a canister before being dispensed. The transmucosal delivery of the supplement matrix is more efficient when the composition is atomized. It allows the enhancer fraction of the composition to adhere more to the nutritional supplement to the mouth's mucosal membranes.

The nutritional supplement matrix used herein can be any combination of electrolytes, vitamins, or minerals. Furthermore, the healthy supplement matrix may be selected from or be a combination of Potassium chloride, Sodium chloride, Iron, Sodium, Calcium, Magnesium, Carbohydrates, Proteins, Zinc, Molybdenum, Caffeine, Copper, Potassium, Manganese, Chlorides, Bicarbonate, and Carbonate, Aluminum, Arsenic, Bromine, Cadmium, Chromium, Sodium, Potassium, Chlorine, Cobalt, Fluorine, Iodine, Citicoline, Tyrosine Phenylalanine, Taurine, Malic Acid, Glucuronolactone, Manganese, Molybdenum Nickel, Phosphorus, Selenium, Silicon, Vanadium, Amino Acids, Vitamin A, Vitamin D, Vitamin E, Vitamin K, Vitamin C, Vitamin B complex, Thiamine (Vitamin 31), Riboflavin (Vitamin 132). Niacin (Vitamin B3), Pyridoxine (Vitamin B6), Biotin, Pantothenic Acid and Pantetheine, Folic Acid, Vitamin B12, “Unofficial” B Vitamins including Choline and Inositol, Vitamin P (bioflavonoids), and flavoring agents, and other vital nutrients, in addition to various homeopathic/alternative substances. In one embodiment, the formulation or composition of nutritional supplement matrix comprises 50 to 60 weight % of Chloride, where the Chloride is from potassium chloride and sodium chloride; 30 to 40 weight % of Sodium, where the Sodium is from sodium chloride; and 5 to 15 weight % of Potassium where the Potassium is from potassium chloride.

The gas fraction used herein comprises ambient air, oxygen, nitrogen or helium. The gas fraction maybe in a compressed state in the canister or in a chamber housed in the canister. In one embodiment, the gas fraction comprises oxygen, wherein the concentration of oxygen is up to 95% oxygen and can mix with other gases such as nitrogen.

The enhancer fraction used herein comprise a mucoadhesive enhancer, an absorption enhancer, and/or a flavoring agent. The mucoadhesive enhancer may comprise a pectin or an apple pectin. The absorption enhancer may be a glycerin or a vegetable glycerin. The flavoring agent may comprise various natural or artificial flavoring agents, allowable to be used in food, nutraceuticals, or pharmaceuticals.

The liquid fraction used herein comprises water, distilled water, filtered water, saline, buffer, or any water-based solutions. The amount of water is sufficient to dissolve all elements in the fraction and prevent precipitating.

The preservative fraction used herein comprise various food-grade or medical-grade preservatives, such as potassium sorbate. The food preservative fraction helps maintain and extend the shelf life of the composition or the sub-compositions.

Transmucosal delivery offers advantages over oral delivery when oral delivery is impossible or negatively impacted by various issues in gastrointestinal tract, stomach, substance digestion and absorption, swallowing, or any other gastrointestinal metabolism problems. The formulation or composition in the present invention may be any form or type of composition suitable for mucosal delivery. Such a composition may comprise a nutraceutical, a pharmaceutical, a food supplement or nutritional supplement.

The following embodiments/examples are included to demonstrate some specifics of the invention. It should be appreciated by those of skill in the art that the techniques disclosed in the examples that follow represent techniques discovered by the inventor to function well in the practice of the present disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the present disclosure.

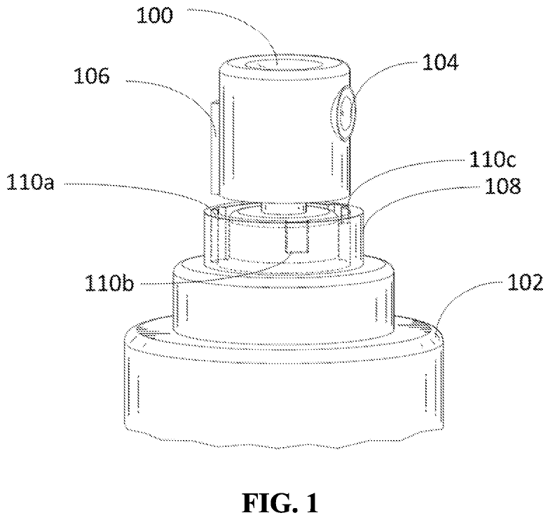

FIG. 1 provides one embodiment of a pump spray cap used in the instant system. The illustrated is an adjustable dose pump spray cap 100, which may be affixed to a canister 102 housing the composition. The system may comprise one or more of the following elements: a nozzle 104, an extension 106, a collar 108, and at least one receiver, such as receivers 110a-110c. The pump spray cap can be in any shape, form or configuration suitable for adjustable dose delivery. A tube may run from the adjustable dose pump spray cap 100 into the canister 102 to draw out the composition housed therein. When the adjustable dose pump spray cap 100 is pressed, certain amount of composition is drawn into the tube, reached the pump spray cap 100, and sprayed out of the nozzle 104. The amount of composition drawn is directly proportional to how far the pump spray cap 100 is pressed. The adjustable dose pump spray cap 100 may have an extension 106. In one embodiment, extension 106 may be a ridge that is a part of the adjustable dose pump spray cap 100. In another embodiment, the system may comprise a collar 108 situated between the adjustable dose pump spray cap 100 and the canister 102. The collar 108 may have at least one receivers, such as receiver 110a, 110b or 110c. The collar 106 and the adjustable dose pump spray cap 100 may rotate independently with one another to allow the user to align the extension 106 with the at least one receiver 110a, 110b or 110c. The receiver 110a-c may be configured to prevent the adjustable dose pump spray cap 100 from being pressed fully. The receiver 110 is set up to restrict the range of motion of the adjustable dose pump spray cap 100. The more restrict of the cap motion, the less amount of the composition is dispensed. For example, 1 ml of the composition may be sprayed out of the nozzle 104 when the adjustable dose pump spray cap 100 is fully pressed one time. In one embodiment, when collar 108 is rotated to align the extension 106 with receiver 110a, the adjustable dose spray cap 100 may be fully pressed. When collar 108 is rotated to align the extension 106 with receiver 110b, the adjustable dose spray cap 110 may be pressed down to two-thirds of its full range of motion, due to an obstruction setup built in the receiver 110b.

By limiting the range of motion of the adjustable dose spray cap 100, the composition dispensed out of the nozzle 104 is reduced from 1 ml. The full range of motion may be achieved by aligning extension 106 with receiver 110a, and 1 ml composition is dispensed. And 0.6 ml composition is dispensed when aligning extension 106 with receiver 110b. In one embodiment, the collar 108 may have a third receiver 110c, allowing the adjustable dose spray cap 100 to be pressed about one-third of its full range of motion. By using receiver 110c, 0.3 ml of the compositions dispensed. This approach may allow users to receive different dose of the composition in a single spray based on their hydration needs and personal preferences. For example, a user may prefer much more frequent doses of the composition, so they may select receiver 110c to receive low dose each time to make sure the composition can last long enough 1. In one embodiment, the composition may come with instructions that recommend dose and dosing frequency based on user's characteristics, such as weight, age, fitness level, and/or characteristics of the user's activity and environment, such as the ambient temperature, expected duration of the activity, location elevation. In one embodiment, the user may have a mobile device, such as a smartphone or wearable smart device, that may make dose and dosing frequency recommendations in real-time based on any of the previously listed characteristics. Such characteristics may be gathered by physiological sensors installed on the user, and communicated, or interpreted by, the mobile device. In one embodiment, collar 108 may have more or less than three receivers.

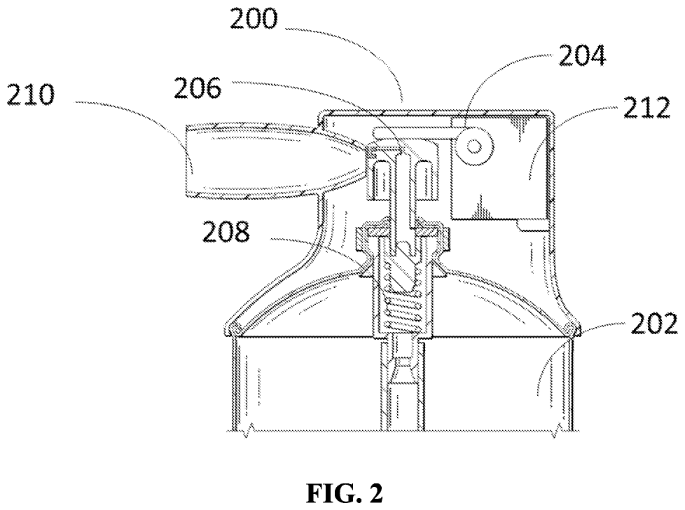

FIG. 2 illustrates a powered aerosol spray cap 200 as part of the instant delivery system. The aerosol spray cap can be in any form, shape or configuration capable of dispensing a composition. The composition may be stored in a canister 202 under pressure. When valve 206 is pressed, the composition may be dispensed through outlet 210. In one embodiment, the composition may be dispensed at a constant and predictable rate when valve 206 is pressed. In one embodiment, the outlet 210 may be shaped to fit in the user's mouth. In one embodiment, outlet 210 may be designed to dispense the composition towards a user's buccal mucosa. When pressure is removed from valve 206, it may be returned to the closed position by a spring 208. It should be obvious that the powered aerosol cap 200 may be affixed to any aerosol valves. Embodiments may include an actuator 204 that may be configured to press the valve 206 when a prompt is received. Embodiments may include an actuator control unit 212 that may have a power source for the actuator, a timer, and an interface that may adjust the time the actuator 204 pressed the valve 206. In one embodiment, the actuator control unit 212 may be communicatively coupled, such as by Bluetooth, to a computing device, such as a smartphone, to allow the user to receive recommendations from a hydration app about the composition dose dispensed with each use. In one embodiment, an interface may be built into the powered aerosol cap 200 that may allow the user to select the amount of the composition dispensed.

For example, 2 ml of the composition may be dispensed when the valve 206 is open for one second. The actuator control unit 212 may have low/med/high dose selections available with a mechanical selector. If the user selects “high,” the actuator 204 may press the valve 206 for 1.0 seconds to dispense 2 ml of the composition. If the user selects “med,” the actuator 204 may press the valve 206 for 0.5 seconds to dispense 1 ml of the composition. If the user selects “low,” the actuator 204 may press the valve 206 for 0.25 seconds to dispense 0.5 ml of the composition. It should be obvious that the time the valve 206 is pressed by the actuator 204 is only limited by the amount of the composition remaining in the canister 202. The dispensed amount maybe in any mount below the full amount housed in the canister 202. In one embodiment, the actuator control unit 212 may be a wheel, to allow a user to select a wider range of delivery dose than just high/med/low.

The foregoing detailed description of the technology has been presented for purposes of illustration and description. It is not intended to be exhaustive or to limit the technology to the precise form disclosed. Many modifications and variations are possible in light of the above teaching. The described embodiments were chosen in order to best explain the principles of the technology and its practical application to thereby enable others skilled in the art to best utilize the technology in various embodiments and with various modifications as are suited to the particular use contemplated. It is intended that the scope of the technology be defined by the claim.

The terms “or” and “and/or,” as used herein, are to be interpreted as inclusive or meaning any one or any combination. Therefore, “A, B or C” or “A, B and/or C” mean any of the following: “A,” “B” or “C”; “A and B”; “A and C”; “B and C”; “A, B and C.” An exception to this definition will occur only when a combination of elements, functions, steps or acts are in some way inherently mutually exclusive.

The terms “comprising,” “including” and “having” are used interchangeably in this disclosure. The terms “comprising,” “including” and “having” mean to include, but not necessarily be limited to the things so described. The terms “about” or “approximately,” as used in the application and claims, should be understood to include the recited values or a value that is three times greater or one third of the recited values.

1. A system for delivering a composition to an oral mucosa, comprising;
i. at least one canister for housing the composition; and
ii. an adjustable dose spray cap comprising a means for allowing the composition to be dispensed in at least two different doses.

2. The system of claim 1, wherein the means comprises a pressable cap with an extension and a rotatable collar.

3. The system of claim 2, wherein the collar comprises at least two different receivers, and wherein each of the at least two different receivers forms an alignment with the extension by rotating the collar.

4. The system of claim 3, wherein the alignment formed with any one of the at least two different receivers enables the pressable cap to be pressed to a different depth, so as to allow the system dispensing at least two different doses of the composition.

5. The system of claim 1, wherein the composition comprises a nutritional composition, a micronutrient composition, a nutraceutical composition, a pharmaceutical composition, a food composition, a food supplement composition, or any combinations thereof.

5. The system of claim 1, wherein the composition comprises any one or any combinations of
(i) a nutritional supplement fraction;
(ii) a gas fraction;
(iii) an enhancer fraction;
(iv) a liquid fraction; or
(v) a preservative fraction.

6. The system of claim 1, wherein the composition comprises a nutritional composition, a micronutrient composition, a nutraceutical composition, a pharmaceutical composition, a food composition, a food supplement composition, or any combinations thereof.

7. A kit comprises:
(i) the system of claim 1;
(ii) an instruction on how to use the system; and
(iii) optionally a smart device, wherein dose or dosing frequency is provided to the system of claim 1.

8. A method of delivering a composition to an oral mucosa, comprising:
(i) obtaining or having obtained a canister, wherein a composition is housed;
(ii) obtaining or having obtained an adjustable dose spray cap, comprising a pressable cap, an extension and a rotatable collar hosting at least one receiver;
(iii) connecting the canister to the adjustable spray cap;
(iv) rotating the collar so that the extension is aligned with the at least one receiver;
(v) pressing the pressable cap so as to dispense a dose of the composition to the oral mucosa.

9. The method of claim 8, wherein the at least one receiver comprises at least two different receivers.

10. The method of claim 9, when in step (iv) rotating the collar to align the extension with a different receiver, a different dose of the composition is released in step (v).

11. A system for delivering a composition to an oral mucosa, comprising;
a. at least one canister for housing the composition; and
b. a powered aerosol spray cap comprising a valve;
wherein the at least one canister is attached to the powered aerosol spray cap; and
wherein the valve is pressed to dispense a dose of the composition to the oral mucosa.

12. The system of claim 11, wherein the powered aerosol spray cap comprises a valve, an actuator and an outlet; and wherein the actuator is configured to press the valve.

13. The system of claim 12, wherein the actuator comprises an actuator control unit;
wherein the actuator control unit comprises a power source for the actuator and a timer; and
wherein the actuator control unit controls the time and/or frequency of the valve being pressed.

14. The system of claim 11, wherein the composition comprises a nutritional composition, a micronutrient composition, a nutraceutical composition, a pharmaceutical composition, a food composition, a food supplement composition, or any combinations thereof.

15. The system of claim 11, wherein the composition comprises any one or any combinations of
(i) a nutritional supplement fraction;
(ii) a gas fraction;
(iii) an enhancer fraction;
(iv) a liquid fraction; or
(v) a preservative fraction.

16. A kit comprises:
(i) the system of claim 11;
(ii) an instruction on how to use the system; and
(iii) a smart device or an interface connectable to the system of claim 11;
wherein the smart device or the interface produces and sends the pre-determined dose information to the system of claim 11.

17-20. (canceled)
